(19) United States
(12) Patent Application Publication
(10) Pub. No.: US 2021/0166928 A1
(43) Pub. Date: Jun. 3, 2021

(54) MASS SPECTROMETRY DATA ANALYSIS DEVICE, MASS SPECTROMETRY DEVICE, METHOD FOR ANALYZING DATA OBTAINED BY MASS SPECTROMETRY AND ANALYSIS PROGRAM

(71) Applicant: SHIMADZU CORPORATION, Kyoto-shi (JP)

(72) Inventors: Hidenori TAKAHASHI, Kyoto-shi (JP); Shinichi Yamaguchi, Kyoto-shi (JP)

(73) Assignee: SHIMADZU CORPORATION, Kyoto-shi (JP)

(21) Appl. No.: 17/071,014

(22) Filed: Oct. 15, 2020

(30) Foreign Application Priority Data
Oct. 16, 2019 (JP) 2019-189377

(51) Int. Cl. H01J 49/00; H01J 49/40; G16C 20/20

(52) U.S. Cl. CPC H01J 49/0036; G16C 20/20; H01J 49/40

(57) ABSTRACT

A device for analyzing data obtained by mass spectrometry, includes: a data acquisition section that acquires mass spectrometry data obtained by first mass spectrometry including subjecting a precursor ion derived from a sample to a reaction using a radical; a first data generation section that generates first data showing two or more candidate structures of a molecule contained in the sample or the precursor ion; a second data generation section that generates second data showing a mass-to-charge ratio or a detected intensity of a product ion generated in a case of subjecting the precursor ion having each of the structures to the reaction; and an information generation section that generates information about identification of the molecule contained in the sample.

### INCORPORATION BY REFERENCE

The disclosure of the following priority application is herein incorporated by reference: Japanese Patent Application No. 2019-189377 filed Oct. 16, 2019.

### TECHNICAL FIELD

The present invention relates to a device for analyzing data obtained by mass spectrometry, a mass spectrometry device, a method for analyzing data obtained by mass spectrometry, and a computer program product.

### BACKGROUND ART

In mass spectrometry, a mass spectrum is obtained which shows peaks corresponding to product ions generated by, for example, dissociation of precursor ions obtained by ionizing a sample, and molecules contained in the sample are analyzed by using this mass spectrum. This method is widely used by, for example, being applied also to imaging mass spectrometry for analyzing molecules present in different positions in a sample (see PTL1 and PTL2).

In such mass spectrometry, for example, dissociation of precursor ions or addition of atoms or atom groups to precursor ions is caused by subjecting the precursor ions to a reaction involving radicals. In this case, it is possible to obtain information about the structure of molecules contained in a sample, such as the amino acid sequence of a protein, by utilizing the specificity of the reaction involving radicals.

In PTL3, oxygen radicals, nitrogen radicals, and the like are generated, and product ions obtained by a reaction between these radicals and fullerene or peptide ions are analyzed. In PTL4, product ions obtained by a reaction between precursor ions derived from phospholipid and radicals are analyzed.

### CITATION LIST

### Patent Literatures

PTL1: Japanese Patent No. 5206790

PTL2: Japanese Patent No. 6222277

PTL3: WO2018/186286

PTL4: WO2019/155725

### SUMMARY OF INVENTION

### Technical Problem

In the analysis of the structure of a molecule contained in a sample based on the characteristics of a reaction using a radical, there is a case where it is difficult to estimate a detailed structure.

### Solution to Problem

The 1st aspect of the present invention relates to a device for analyzing data obtained by mass spectrometry, comprising: a data acquisition section that acquires mass spectrometry data obtained by first mass spectrometry including subjecting a precursor ion derived from a sample to a reaction using a radical; a first data generation section that generates, on a basis of sample information about a molecule contained in the sample, first data showing two or more candidate structures of the molecule or the precursor ion; a second data generation section that generates, on a basis of the first data and data showing a condition of the reaction, second data showing at least one of a mass-to-charge ratio and a detected intensity of a product ion generated in a case of subjecting the precursor ion having each of the structures to the reaction; and an information generation section that generates, on a basis of at least one of a mass-to-charge ratio and a detected intensity of a detected product ion in the mass spectrometry data and the second data, information about identification of the molecule contained in the sample.

The 2nd aspect of the present invention relates to a mass spectrometry device comprising: a measurement unit that performs first mass spectrometry including subjecting a precursor ion derived from a sample to a reaction using a radical; and the device for analyzing data obtained by mass spectrometry according to the 1st aspect.

The 3rd aspect of the present invention relates to a method for analyzing data obtained by mass spectrometry, comprising: acquiring mass spectrometry data obtained by first mass spectrometry including subjecting a precursor ion derived from a sample to a reaction using a radical; generating, on a basis of sample information about a molecule contained in the sample, first data showing two or more candidate structures of the molecule or the precursor ion; generating, on a basis of the first data and data showing a condition of the reaction, second data showing at least one of a mass-to-charge ratio and a detected intensity of a product ion generated in a case of subjecting the precursor ion having each of the structures to the reaction; and generating, on a basis of at least one of a mass-to-charge ratio and a detected intensity of a detected product ion in the mass spectrometry data and the second data, information about identification of the molecule contained in the sample.

The 4th aspect of the present invention relates to a computer-readable computer program product containing an analysis program for allowing a computer to perform: data acquisition processing to acquire mass spectrometry data obtained by first mass spectrometry including subjecting a precursor ion derived from a sample to a reaction using a radical; first data generation processing to generate, on a basis of sample information about a molecule contained in the sample, first data showing two or more candidate structures of the molecule or the precursor ion; second data generation processing to generate, on a basis of the first data and data showing a condition of the reaction, second data showing at least one of a mass-to-charge ratio and a detected intensity of a product ion generated in a case of subjecting the precursor ion having each of the structures to the reaction; and information generation processing to generate, on a basis of at least one of a mass-to-charge ratio and a detected intensity of a detected product ion in the mass spectrometry data and the second data, information about identification of the molecule contained in the sample.

### Advantageous Effects of Invention

According to the present invention, it is possible to analyze the structure of a molecule contained in a sample in more detail on the basis of the characteristics of a reaction using a radical.

### BRIEF DESCRIPTION OF DRAWINGS

FIG. 1 is a conceptual diagram showing the structure of a mass spectrometry device according to an embodiment.

FIG. 2 is a conceptual diagram showing the structure of an information processing unit according to the embodiment.

FIG. 3 is a conceptual diagram for explaining a method for analyzing data obtained by mass spectrometry according to the embodiment.

FIG. 4 is a flow chart showing the procedure of a method for analyzing data obtained by mass spectrometry according to the embodiment.

FIG. 5 is a conceptual diagram for explaining a method for analyzing data obtained by mass spectrometry according to a Variation.

FIG. 6 is a flow chart showing the procedure of a method for analyzing data obtained by mass spectrometry according to the Variation.

FIG. 7 is a conceptual diagram showing the structure of an information processing unit according to a Variation.

FIG. 8 is a conceptual diagram for explaining a method for analyzing data obtained by mass spectrometry according to the Variation.

FIG. 9 is a conceptual diagram showing the structure of a mass spectrometry device according to a Variation.

FIG. 10 is a conceptual diagram for explaining the supply of a program.

FIG. 11 is a graph obtained in an Example, which shows the similarity of a candidate structure and the number of candidate structures having each similarity value.

FIG. 12 shows a mass spectrum (a) obtained by measurement in an Example and a product ion spectrum (b) corresponding to a calculated candidate structure and having the highest similarity.

### DESCRIPTION OF EMBODIMENT

Hereinbelow, an embodiment for carrying out the present invention will be described with reference to the drawings. In the embodiment that will be describe below, the term “product ion” refers not only to an ion generated by dissociation of a precursor ion but also to an ion generated not by dissociation but by a reaction between a precursor ion and a radical. For example, the term “product ion” refers also to an adduct ion generated by addition of an atom or an atom group to a precursor ion.

### First Embodiment

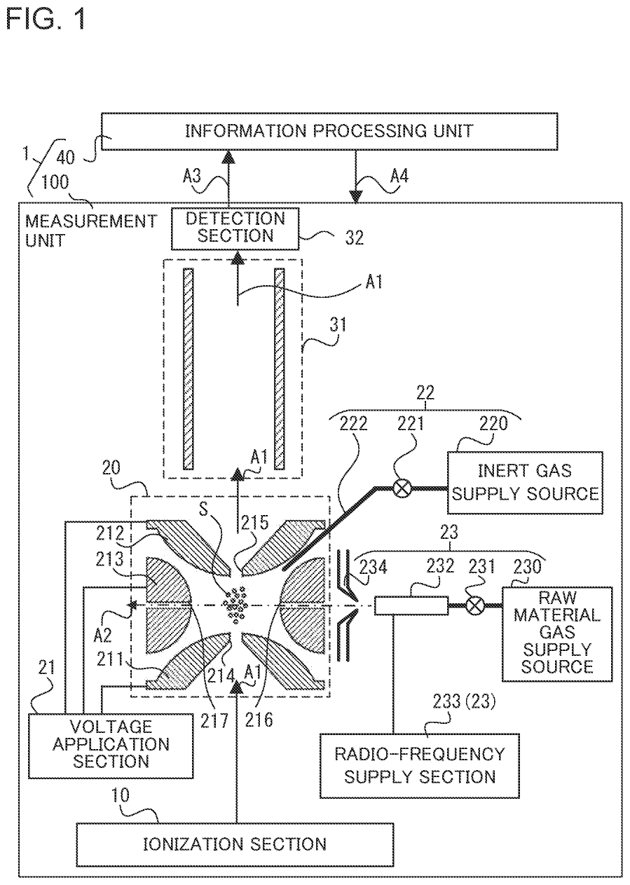

FIG. 1 is a conceptual diagram showing the structure of a mass spectrometry device 1 according to the present embodiment. The mass spectrometry device 1 includes a measurement unit 100 and an information processing unit 40. The information processing unit 40 constitutes a device for analyzing data obtained by mass spectrometry according to the present embodiment.

The measurement unit 100 includes an ionization section 10, an ion generation section 20 that traps a sample-derived ion S, a voltage application section 21, an inert gas supply section 22, a radical supply section 23, a time-of-flight mass separation section 31, and a detection section 32.

The ion generation section 20 includes an inlet-side end cap electrode 211, an outlet-side end cap electrode 212, a ring electrode 213, an ion introduction port 214, an ion ejection port 215, a radical introduction part 216, and a radical discharge part 217. The inert gas supply section 22 includes an inert gas supply source 220, a valve 221, and an inert gas introduction part 222. The radical supply section 23 includes a raw material gas supply source 230, a valve 231, a radical generation part 232, a radio-frequency supply part 233, and a radical separation part 234. In FIG. 1, the migration path of the sample-derived ions S to be detected is schematically shown by arrows A1, and the flow of a detection signal generated by detection of the sample-derived ion S is schematically shown by an arrow A3. The control of the measurement unit 100 performed by the information processing unit 40 is schematically shown by an arrow A4. Further, the migration path of a radical supplied from the radical supply section 23 is shown by an arrow A2.

The measurement unit 100 performs mass spectrometry including subjecting a precursor ion generated by ionizing a sample to a reaction using a radical. This mass spectrometry refers to first mass spectrometry.

The ionization section 10 of the measurement unit 100 includes an ion source and ionizes a molecule contained in the sample. A method used for ionization is not particularly limited, and may be matrix-assisted laser desorption/ionization (MALDI), electrospray ionization (ESI), or the like. When an ion source for MALDI is used, the ion source of the ionization section 10 includes a sample plate holder (not shown) that supports a sample plate for MALDI and a laser (not shown) that irradiates the sample plate for MALDI with laser light. In this case, the ionization section 10 performs ionization by irradiating a crystal of a sample and a matrix, prepared from the sample, with laser light.

It is to be noted that the mass spectrometry device 1 may further include a separation analysis device such as a gas chromatograph (GC) or a liquid chromatograph (LC) to ionize a sample eluted from the GC or LC in the ionization section 10. In this case, the number of peaks in a mass spectrum can be reduced by separation performed by the separation analysis device, and therefore analysis can be performed more accurately. That is, the mass spectrometry device 1 may be a gas chromatograph-mass spectrometer or a liquid chromatograph-mass spectrometer.

The sample is not particularly limited as long as the molecule contained in the sample can be ionized. The device for analyzing data obtained by mass spectrometry according to the present embodiment is suitable for distinguishing between/among, for example, two or more isomers different in the length of carbon chains or the position of a double bond in a carbon chain. Therefore, it is preferred that the sample contains or may contain a lipid, and it is more preferred that the sample contains or may contain a lipid containing two or more fatty acids. In the embodiment that will be described below, the term “lipid” refers to a biological substance containing a fatty acid or a hydrocarbon chain. Here, a molecule in which a fatty acid is present as an acyl group is also regarded as a molecule containing a fatty acid.

In the embodiment that will be described below, sample-derived ions S include not only an ion generated by ionization of the sample performed by the ionization section 10 but also a product ion generated by a reaction between a precursor ion and a radical. Therefore, the sample-derived ions S include a fragment ion generated by dissociation of a precursor ion, an ion generated by, for example, binding of an atom or an atom group to precursor ion, and the like. Examples of the latter include an adduct ion generated by addition of an atom or an atom group to a precursor ion. The sample-derived ion S generated by ionization performed by the ionization section 10 are introduced into the ion generation section 20 through the ion introduction port 214.

The ion generation section 20 includes a vacuum chamber capable of trapping an ion, such as an ion trap, and generates a product ion by reacting the sample-derived ion S with a radical introduced into the ion generation section 20. The sample-derived ion S to be subjected to the reaction with the radical is referred to as a precursor ion. The precursor ion is preferably selected by mass separation, but when the composition of the sample is not complicated, mass separation of the precursor ion is not always necessary. In the ion generation section 20, the inlet-side end cap electrode 211 having the ion introduction port 214 formed therein faces the outlet-side end cap electrode 212 having the ion ejection port 215 formed therein with the circular ring electrode 213 being interposed therebetween. The ion generation section 20 controls the sample-derived ions S by voltages applied by a voltage application part 21 to the end cap electrodes 211 and 212 and the ring electrode 213 and a cooling gas or the like introduced so that the sample-derived ions S are appropriately trapped or discharged.

It is to be noted that the structure of the ion generation section 20 is not particularly limited as long as the sample-derived ions S can be trapped. The ion generation section 20 may have a structure including a three-dimensional ion trap shown in FIG. 1 as an example, a linear ion trap, a collision cell, or the like. When the ion generation section 20 is not an ion trap and does not have a structure capable of appropriately performing mass separation, a mass analyzer such as a quadrupole is preferably provided before the ion generation section 20.

The radical reaction caused in the ion generation section 20 is not particularly limited as long as it is a reaction using a radical. Hereinbelow, the radical reaction refers to a radical reaction that occurs in the ion generation section 20 unless otherwise specified. From the viewpoint of reducing the number of product ions as candidates to more efficiently analyze the molecule contained in the sample, the radical reaction preferably has specificity for a position in the precursor ion where dissociation or binding with an atom or an atom group occurs.

For example, dissociation of the precursor ion can be caused by attachment of a hydrogen radical to the sample-derived ion S or abstraction of hydrogen from the sample-derived ion S by a hydrogen radical. When such dissociation occurs, the peptide main chain of a peptide ion is cleaved at a specific position. In a reaction between a hydroxy radical or an oxygen radical that is a radical having oxidizing ability and the precursor ion having a hydrocarbon chain, the precursor ion is selectively dissociated at the position of an unsaturated bond in the hydrocarbon chain. At this time, an oxygen atom may be added to a carbon atom that formed the unsaturated bond so that a fragment ion is generated. In a reaction between a nitrogen radical that is a radical having reducing ability and the precursor ion having a hydrocarbon chain, the precursor ion is dissociated at the position of a carbon-carbon bond of the hydrocarbon chain irrespective of whether the carbon-carbon bond is a saturated bond or an unsaturated bond so that a fragment ion is generated.

In FIG. 1, the voltage application section 21 includes a voltage generator capable of generating a voltage that periodically changes, and applies voltages to the end cap electrodes 211 and 212 and the ring electrode 213 to control the movement of the sample-derived ions S introduced into the ion generation section 20. The sample-derived ions S are trapped in the ion generation section 20 by voltage control performed by the voltage application section 21. When the ion generation section 20 is an ion trap, a precursor ion is mass-separated from the sample-derived ions S by voltage control performed by the voltage application section 21 so that some of the ions are discharged from the ion generation section 20. The product ions generated in the ion generation section 20 are discharged into the time-of-flight mass separation section 31 through the ion ejection port 215.

The inert gas supply section 22 supplies a cooling gas to the ion generation section 20. The inert gas supply source 220 includes a cooling gas storage container (not shown) containing a cooling gas such as helium or argon. The composition of the cooling gas is not particularly limited. The introduction of the cooling gas is controlled by opening and closing of the valve 221 provided in the middle of a conduit for such a gas and controlled by a device control part 51 that will be described later. The inert gas introduction part 222 includes a conduit extending to the ion generation section 20 and introduces an inert gas into the ion generation section 20.

It is to be noted that when the mass spectrometry device 1 performs second mass spectrometry that will be described later, a CID gas for collision-induced dissociation (CID) can be introduced from the inert gas supply section 22.

The radical supply section 23 supplies a radical to the ion generation section 20. The raw material gas supply source 230 includes a storage container (not shown in the figure) for a raw material gas containing a molecule to be converted into a radical.

It is to be noted that the raw material gas is selected on the basis of the kind of radicals to be generated by the radical generation part 232. The raw material gas may contain, for example, at least one of nitrogen, oxygen, hydrogen, hydrogen peroxide, and water vapor. For example, when the raw material gas is nitrogen gas, a nitrogen radical is generated. When the raw material gas is oxygen gas, an oxygen radical is generated. When the raw material gas is hydrogen gas, a hydrogen radical is generated. When the raw material gas is hydrogen peroxide gas, an oxygen radical and a hydrogen radical are generated. When the raw material gas is air, an oxygen radical, a nitrogen radical, or the like are generated. When the raw material gas is water vapor, a hydroxyl radical, an oxygen radical, and a hydrogen radical are generated.

The discharge of the gas from the raw material gas supply source 230 and the flow rate of the gas are controlled by opening and closing of the valve 231 provided in the middle of a conduit for the raw material gas and controlled by the device control part 51 that will be described later. The gas that has passed through the valve 231 is introduced into the radical generation part 232.

The radical generation part 232 includes a radical generation chamber into which the raw material gas is introduced. The radical generation chamber is evacuated by a vacuum pump (not shown in the figure) to a pressure at which vacuum discharge can be performed. The radical generation part 232 has a structure in which electrodes are provided so that vacuum discharge occurs in the radical generation chamber and microwaves from the radio-frequency supply part 233 are supplied to the electrodes. When vacuum discharge occurs in the radical generation chamber, the raw material gas is converted into plasma by vacuum discharge so that a radical is generated. The radical generation part 232 may be an inductively coupled radio-frequency radical source, but for example, a method for generating plasma is not particularly limited. The radical generated in the radical generation part 232 is jetted through a nozzle or the like (not shown in the figure) into the radical separation part 234.

It is to be noted that a method for generating a radical is not particularly limited to a method using electric discharge, such as plasma generation. For example, oxygen atoms may be generated by heating a platinum oxide filament to about 1000° C.

The radio-frequency supply part 233 includes a microwave supply source and a matching box such as a three-stub tuner, and supplies, to the radical generation part 232, microwaves generated by the microwave supply source and subjected to impedance matching by the matching box.

The radical separation part 234 includes a structure, such as a skimmer, which allows some of molecules from the radical generation part 232 to pass through it. The radical separation part 234 removes at least part of the raw material gas that has not been converted into a radical and allows a radical to pass through it so that the radical is introduced into the radical introduction part 216. The radical introduction part 216 includes an orifice formed in the ring electrode 213 and introduces the introduced radical into the ion generation section 20. The sample-derived ions S are irradiated with the radical introduced into the ion generation section 20.

The radical discharge part 217 is formed on a straight line that is substantially the same as the long axis of the orifice of the radical introduction part 216, and discharges a radical that has not interacted with the sample-derived ions S, etc. to prevent a reduction in the degree of vacuum.

It is to be noted that a method for generating and introducing a radical is not particularly limited as long as a radical can be generated at a flow rate necessary for allowing a data processing part 52 that will be described later to perform analysis with desired accuracy.

The time-of-flight mass separation section 31 includes a vacuum chamber, such as a flight tube, in which ions fly, and separates the product ion accelerated after ejection from the ion ejection port 215 on the basis of time-of-flight. The type of the time-of-flight mass separation section 31 is not particularly limited, and a multi-turn type, a reflectron type, or the like may appropriately be used instead of a linear type shown in the drawing.

It is to be noted that the configuration of a mass spectrometry section that performs a radical reaction and one- or more-step mass separation is not particularly limited as long as the product ion generated in the ion generation section 20 can be separated at a desired resolution and detected. For example, mass separation may be performed by resonance excitation ejection by an ion trap or the like of the ion generation section 20. For example, when mass separation is not performed in the ion generation section 20, the precursor ion and the product ion may be mass-separated by providing any one or more mass analyzers before and after the ion generation section 20, respectively.

The detection section 32 includes an ion detector such as a microchannel plate, and detects the product ion separated by the time-of-flight mass separation section 31 on the basis of time-of-flight. Detection signals obtained by detection performed by the detection section 32 are A/D converted by an analog/digital (A/D) converter (not shown in the figure) and outputted to the information processing unit 40. Hereinafter, data obtained from detection signals obtained by detection performed by the detection section 32 is referred to as measured data.

The information processing unit 40 includes an information processing device such as a computer, and appropriately serves as an interface with a user of the mass spectrometry device 1 (hereinafter simply referred to as “user”) and performs processing such as communication, storage, and calculation of various data.

It is to be noted that the information processing unit 40 may be configured as a single device integrated with the measurement unit 100. Further, part of data used by the mass spectrometry device 1 may be stored in a remote server or the like.

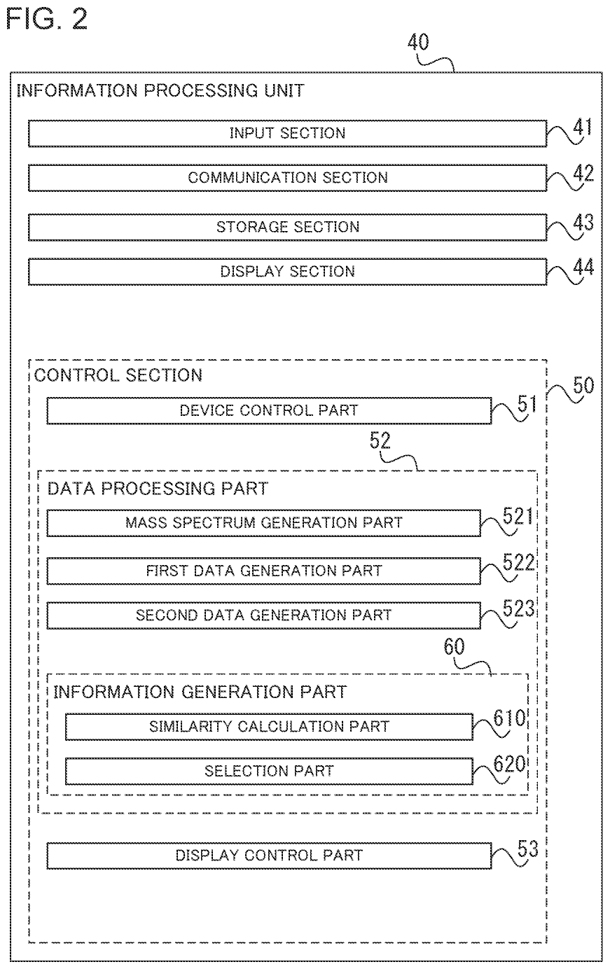

FIG. 2 is a conceptual diagram showing the structure of the information processing unit 40. The information processing unit 40 includes an input section 41, a communication section 42, a storage section 43, a display section 44, and a control section 50. The control section 50 includes a device control part 51, a data processing part 52, and a display control part 53. The data processing part 52 includes a mass spectrum generation part 521, a first data generation part 522, a second data generation part 523, and an information generation part 60. The information generation part 60 includes a similarity calculation part 610 and a selection part 620.

The input section 41 of the information processing unit 40 includes an input device such as a mouse, a keyboard, various buttons, or a touch panel. The input section 41 receives, from the user, information necessary for control of operation of the measurement unit 100, information necessary for processing performed by the control section 50, etc.

The communication section 42 of the information processing unit 40 includes a communication device that enables communication through a network such as the Internet by wireless or wired connection. The communication section 42 sends and receives data as necessary.

The storage section 43 of the information processing unit 40 includes a non-volatile storage medium, and stores analysis conditions, measured data, programs for allowing the control section 50 to execute processing, etc.

The storage section 43 stores sample information that is information about the molecules contained in the sample. The sample information may be acquired through the input section 41 or the communication section 43 or may be generated from information obtained by second mass spectrometry (to be described later) performed by the mass spectrometry device 1. The sample information includes the kind of the molecules contained in the sample. Here, the kind of molecules is preferably a classification based on the structure of molecules, such as lipid, phospholipid, or protein. The sample information may further include various information for partially identifying the structure of the molecules contained in the sample.

When the sample contains a lipid, the sample information may include information about the class or head group of the lipid, the length of carbon chain of a fatty acid contained in the lipid, the number of double bonds in the carbon chain, and a modification group in the carbon chain. The length of the carbon chain is preferably expressed as, for example, the number of carbon atoms constituting the carbon chain.

The class of the lipid refers to a classification based on the structure of a moiety other than a fatty acid in the lipid. For example, phospholipids are divided into classes such as phosphatidylcholine, phosphatidylethanolamine, phosphatidylglycerol, phosphatidylinositol, phosphatidylserine, and sphingomyelin. These classes are often classifications based on the structure of head group of lipid, and therefore the sample information may include information about head group as a replacement for information about class or as additional information.

The storage section 43 stores a database (not shown in the figure). The database includes data about the structures of molecules. This data is used for generating candidate structure data or candidate product data that will be described later. This data includes, for example, information about the masses of moieties in molecules, such as carbon chains, functional groups, or head groups. Further, this data may include information about a position in the moiety where cleavage is likely to occur by dissociation using a radical, a binding position in the moiety to which an atom or an atom group is bound in a radical reaction, and the mass of an atom or an atom group bound to the binding position when a predetermined radical is used. This data may include information about the detected intensity of product ions or the like to be generated. Here, the detected intensity is a value indicating the magnitude of a detection signal obtained by detection of ions, and may be, for example, a peak area that is the area of a peak in a mass spectrum or a peak intensity that is the largest intensity of a peak in a mass spectrum.

The display section 44 of the information processing unit 40 includes a display device such as a liquid crystal monitor, and displays, on the display device, information obtained by processing performed by the data processing part 52, etc.

The control section 50 of the information processing unit 40 includes a processor such as a central processing unit (CPU) and a storage medium such as a memory, and functions as a main part of operation to control the mass spectrometry device 1. The control section 50 serves as a processing device that performs analytical processing of data obtained by first mass spectrometry, etc. The control section 50 performs various types of processing by loading a program stored in the storage section 43 or the like into the memory and allowing the processor to execute the program.

It is to be noted that the physical structure, etc. of the control section 50 are not particularly limited as long as the processing by the control section 50 according to the present embodiment can be performed.

The device control part 51 of the control section 50 controls the operation of the measurement unit 100 on the basis of information about analysis conditions inputted via the input section 41 or information stored in the storage section 43. The device control part 51 controls voltages applied by the voltage application section 21, introduction of an inert gas and radicals into the ion generation section 20, detection performed by the detection section 32, etc.

The data processing part 52 of the control section 50 performs analytical processing of measured data.

It is to be noted that the device for analyzing data obtained by mass spectrometry according to the present embodiment may also be configured as an analysis device that includes the data processing part 52 and does not perform device control.

The mass spectrum generation part 521 of the data processing part 52 generates data corresponding to a mass spectrum from measured data. Hereinafter, the data corresponding to a mass spectrum is referred to as mass spectrum data. The mass spectrum generation part 521 functions as a data acquisition part that acquires measured data outputted from the detection section 32 and stores the measured data in, for example, the memory or the storage section 43. The mass spectrum generation part 521 generates mass spectrum data by a method appropriate to the method of mass separation. For example, when the sample-derived ions S are detected by time-of-flight mass spectrometry, the measured data includes the intensities of detection signals at different times-of-flight. The mass spectrum generation part 521 converts the times-of-flight into m/z values using calibration data previously acquired and generates mass spectrum data by associating the m/z values with the intensities. Further, the mass spectrum generation part 521 calculates, from the mass spectrum data of product ions, at least one of the m/z value and the detected intensity of each kind of product ions, and stores it in the storage section 43 or the like as measured product data. Hereinbelow, m/z is used as a mass-to-charge ratio, but the mass-to-charge ratio is not particularly limited as long as it represents the ratio of mass to charge of an ion.

Hereinafter, a mass spectrum obtained by mass separation of a product ion obtained by a radical reaction is referred to as product ion spectrum. In the embodiment that will be described below, the term “product ion spectrum” also refers to a spectrum containing the peak of an ion other than ions generated by dissociation, such as an adduct ion. The mass spectrum generation part 521 generates mass spectrum data corresponding to a product ion spectrum and stores it in the storage section 43 or the like.

The first data generation part 522 of the data processing part 52 generates data showing two or more candidate structures of a precursor ion derived from the sample. Hereinafter, this data is referred to as candidate structure data, and candidates for the structure of a molecule contained in the sample are referred to as candidate structures.

It is to be noted that the first data generation part 522 may generate data showing two or more candidate structures of a molecule contained in the sample. Also in this case, the data processing part 52 can perform substantially the same processing.

The first data generation part 522 calculates candidate structures on the basis of sample information. The first data generation part 522 calculates, from the m/z value of the precursor ion, two or more candidate structures having a m/z value within the range of variation of m/z in mass spectrometry, such as a mass tolerance. The first data generation part 522 can further limit the calculated candidate structures on the basis of sample information. From the viewpoint of limiting the number of candidate structures for efficient data analysis, the m/z value of the precursor ion is preferably obtained precisely, more preferably obtained with a mass resolution of 10 ppm or less, even more preferably obtained with a mass resolution of 1 ppm or less.

For example, when the sample information indicates that the sample contains a phospholipid, the first data generation part 522 can calculate the structures of two or more isomers different in the position of a double bond as candidate structures. Alternatively, the first data generation part 522 can calculate, as candidate structures, the structures of two or more isomers in which a part of fatty acids contained in the precursor ion have a shorter carbon chain and another part of fatty acids have a longer carbon chain. The first data generation part 522 can calculate two or more structures different in mass other than isomers as data included in the candidate structure data as long as the m/z values of the candidate structures are within the range of variation of m/z of the precursor ion based on mass precision. The first data generation part 522 refers to the database in the storage section 43 as necessary to acquire the masses of atoms, functional groups, etc. when calculating these candidate structures.

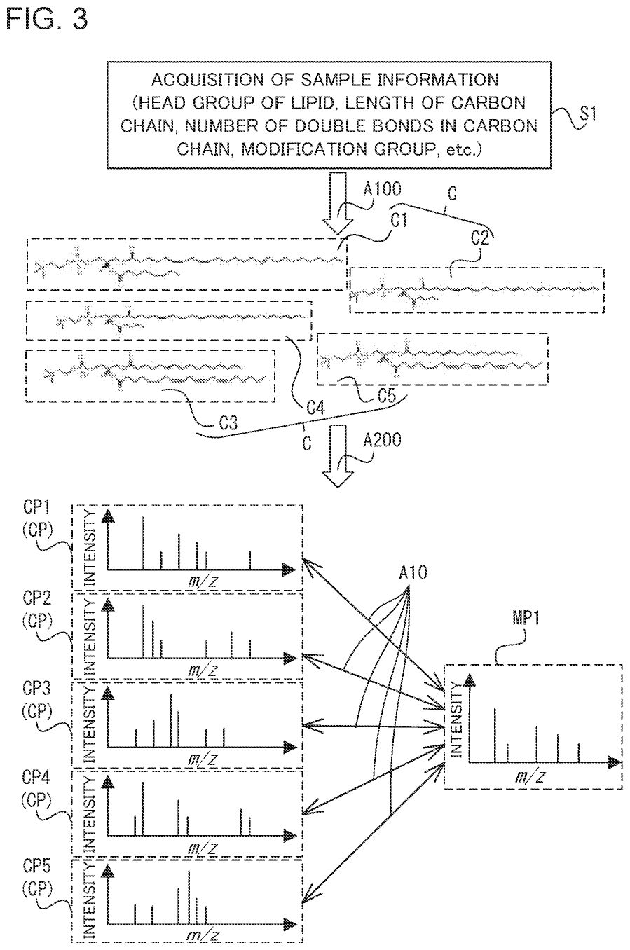

FIG. 3 is a conceptual diagram for explaining a method for analyzing data obtained by mass spectrometry according to the present embodiment. The first data generation part 522 calculates two or more candidate structures C (arrow A100) after acquiring sample information (column S1). In the example shown in FIG. 3, five candidate structures C1 to C5 are shown as candidate structures C, but the number of candidate structures C to be calculated is not particularly limited. A method for expressing the candidate structures C as candidate structure data is not particularly limited.

The second data generation part 523 of the data processing part 52 generates data showing at least one of the m/z value and the detected intensity of product ions generated by subjecting a precursor ion derived from the sample having each of the calculated candidate structures C to a radical reaction. This data is referred to as candidate product data. The candidate product data may include the m/z value(s) or detected intensity (intensities) of one or more kinds of product ions generated by subjecting the precursor ion having the candidate structure C corresponding to each of the candidate structures C to a radical reaction. From such a viewpoint, the candidate product data can be said also as data corresponding to an estimated product ion spectrum.

The second data generation part 523 generates candidate product data on the basis of candidate structure data and data showing the conditions of a radical reaction. Hereinafter, the data showing a condition of a radical reaction is referred to as reaction condition data. The reaction condition data is acquired through the input section 41 or the communication section 42. The reaction condition data includes the kind of radicals involved in the radical reaction, such as radicals introduced into the ion generation section 20, etc. The second data generation part 523 refers to the database in the storage section 43 as necessary to acquire the cleavage position in dissociation and the mass of an atom or an atom group to be added, and generates candidate reactant data. The detected intensity in the candidate product data can be acquired by referring to the database in the storage section 43.

The information generation part 60 generates information about the identification of the molecule contained in the sample on the basis of at least one of the m/z value and the detected intensity, obtained from measured data, of a product ion detected by the detection section 32 and candidate product data. This information is referred to as identification information.

The similarity calculation part 610 of the information generation part 60 calculates a similarity between the m/z value or detected intensity in candidate product data corresponding to each of the candidate structures C and the m/z value or detected intensity of the detected product ion. When the m/z value of a detected product ion falls within the range of variation of m/z in the candidate product data based on mass precision, the similarity is set to be higher. Further, in such a case, when the difference between the value of the detected intensity in the candidate product data and the value of the detected intensity of the detected product ion is within a predetermined range, the similarity is set to be even higher. The comparison of detected intensity is preferably performed using a ratio determined by dividing the detected intensity of a product ion by the detected intensity of a precursor ion in the product ion spectrum. In this case, the above predetermined range can be set to, for example, 0.2 or less, preferably 0.1 or less.

It is to be noted that a method for defining the similarity or a method for calculating the similarity is not particularly limited, and for example, a method performed in spectrum matching in mass spectrometry may be used.

In FIG. 3, generating candidate product data CP and calculating a similarity after the candidate structure data is generated are schematically shown by an arrow A200. The similarity calculation part 610 refers to measured product data generated by the mass spectrum generation part 521 and including the m/z value and detected intensity of each kind of product ions. In FIG. 3, this data including the m/z values and the detected intensities obtained by calculation is schematically shown as measured product data MP1. In FIG. 3, calculating a similarity between each of the candidate product data CP and the measured product data MP1 by the similarity calculation part 610 and comparing these data on the basis of the similarity by the information generation part 60 are schematically shown by arrows A10. In FIG. 3, five candidate product data CP1 to CP5 are shown, but the number of candidate product data CP to be generated or compared is not particularly limited.

The selection part 620 of the information generation part 60 selects the structure of the molecule contained in the sample among the candidate structures C on the basis of the similarity calculated by the similarity calculation part 610. The selection part 620 can set a molecule having the candidate structure C corresponding to the candidate product data CP having the highest similarity as the molecule contained in the sample. It is to be noted that the selection part 620 does not always need to select only one candidate structure C. The selection part 620 may select any number of candidate structures C by, for example, selecting several candidate structures C in order of decreasing the similarity.

The information generation part 60 generates information showing a molecule having the candidate structure C selected by the selection part 620 as identification information. A method for expressing the identification information is not particularly limited as long as the molecule can be identified. For example, the identification information may include the name, composition, structural formula, etc. of the molecule. The identification information may include the similarity. The form of the identification information is not particularly limited, and the identification information can be expressed by various methods such as sentences and images.

The display control part 53 controls the display section 44 to display identification information. The display control part 53 can display information about analysis conditions or any information obtained by mass spectrometry together with identification information. For example, the display control part 53 can display both identification information and a product ion spectrum. Further, when the selection part 620 selects two or more candidate structures C, the display control part 53 can display them in list form in the order of decreasing the similarity.

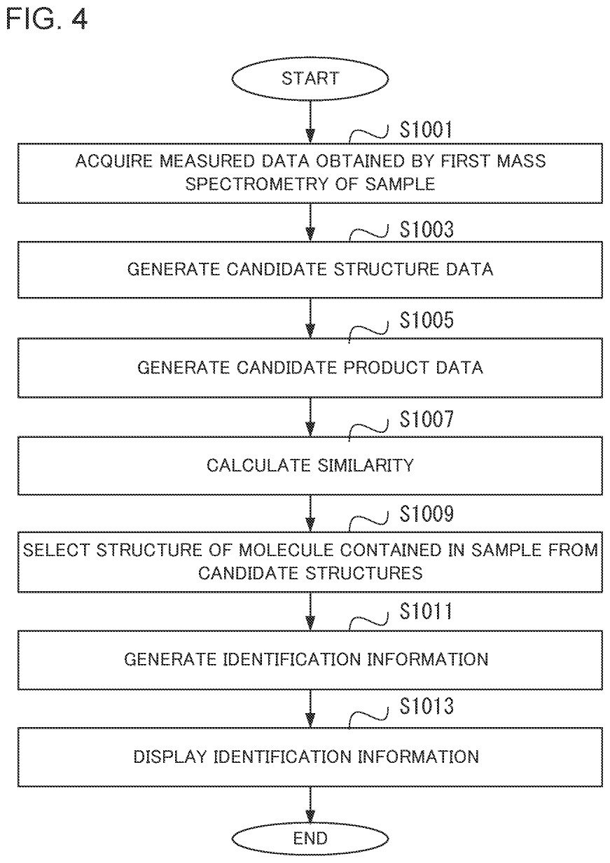

FIG. 4 is a flow chart showing the procedure of the method for analyzing data obtained by mass spectrometry according to the present embodiment. Each step shown in FIG. 4 is appropriately performed by the control section 50. In step S1001, the mass spectrum generation part 521 acquires measured data obtained by first mass spectrometry of a sample. After step S1001 is completed, step S1003 is started.

In step S1003, the first data generation part 522 generates candidate structure data. After step S1003 is completed, step S1005 is started. In step S1005, the second data generation part 523 generates candidate product data. After step S1005 is completed, step S1007 is started.

In step S1007, the similarity calculation part 610 calculates a similarity between the m/z value or the like of measured product ions and the m/z value or the like of product ions corresponding to each candidate structure C. After step S1007 is completed, step S1009 is started. In step S1009, the selection part 620 selects the structure of the molecule contained in the sample from two or more candidate structures C. After step S1009 is completed, step S1011 is started.

In step S1011, the information generation part 60 generates identification information. After step S1011 is completed, step S1013 is started. In step S1013, the display control part 53 displays the identification information. After step S1013 is completed, the processing is completed.

The following variations are within the scope of the present invention, and may be combined with the above-described embodiment. In the following Variations, sites similar in structure and function to those of the above-described embodiment are denoted by the same reference numerals, and description thereof will be omitted as appropriate.

Variation 1

In the above-described embodiment, when a molecule contained in the sample includes a double bond, the data processing part 52 may be configured to distinguish whether the double bond is of a cis type or a trans type.

As shown in PTL4, there is a case where the detected intensity of an adduct ion having an oxygen atom attached by a radical reaction depends on whether a double bond of a carbon chain of a precursor ion of a lipid is of a cis type or a trans type.

In the database of the storage section 43, the ratio of the detected intensity of an adduct ion to the detected intensity of a precursor ion when a cis type double bond is present under a specific condition and that when a trans type double bond is present under the specific condition are stored on the basis of, for example, measurement previously performed. The specific condition refers to a condition relating to the kind or structure of a molecule, and for example, refers to a condition that the carbon chain of a fatty acid of a lipid contained in the sample has a double bond. The first data generation part 522 can calculate, from the m/z value of a precursor ion and sample information, a cis-type candidate structure C and a trans-type candidate structure C which satisfy the above specific condition. In this case, the second data generation part 523 calculates, from the above ratio in the database, the detected intensity of an adduct ion in candidate product data CP corresponding to the cis-type candidate structure C and the detected intensity of an adduct ion in candidate product data CP corresponding to the trans-type candidate structure C. These detected intensities are different from each other, and therefore the similarity calculation part 610 calculates different similarities for the cis type and the trans type. As a result, a distinction between the cis type and the trans type can be made on the basis of the detected intensity of a detected adduct ion. PTL4 states that the detected intensity of a trans-type adduct ion is higher than that of a cis-type adduct ion. However, a distinction can be made also when the detected intensity of a cis-type adduct ion is higher than that of a trans-type adduct ion as long as information is stored in the database on the basis of previously-obtained measurement results. As described above, there is a case where the structure of the molecule contained in the sample can be identified not on the basis of a difference in m/z but on the basis of only a difference in detected intensity.

It is to be noted that the detected intensity of a fragment ion tends to be higher as the distance between the position of cleavage in a carbon chain of a fatty acid or the like of a lipid in dissociation and the terminal of the carbon chain increases. By reflecting such a difference in detected intensity in candidate product data CP, the structure of a lipid contained in the sample can more accurately be identified.

Variation 2

In the above-described embodiment, the sample information may be acquired by second mass spectrometry performed before first mass spectrometry. The second mass spectrometry may be performed by the mass spectrometry device 1 or a mass spectrometry device other than the mass spectrometry device 1.

The method of the second mass spectrometry is not particularly limited as long as information about the structure of the molecule contained in the sample can be obtained. For example, the second mass spectrometry is performed by two- or more-step mass spectrometry including dissociation by CID. This makes it possible to obtain information about the internal structure of the molecule contained in the sample by using dissociation. For example, when the sample contains a lipid, cleavage is performed at a position near or in a head group, and therefore it is possible to obtain information about the head group and the class of the lipid. Further, the two- or more-step mass spectrometry using CID makes it possible to obtain information about the length of a carbon chain, the number of double bonds, the kind of a modification group, etc.

It is to be noted that in the second mass spectrometry, dissociation may be performed by any method other than CID, and dissociation using a radical may be performed. Alternatively, the second mass spectrometry may be performed by one-step mass spectrometry.

Variation 3

In the above-described embodiment, there is a case where two or more kinds of precursor ions are mass-separated by mass separation of precursor ions in first mass spectrometry. The reason for this is because irrespective of the mass resolution of the mass spectrometry device 1, it is necessary to set a margin for the m/z value of a precursor ion to be mass-separated to about 0.5 to 3 in order to achieve sensitivity necessary for a precursor ion having a target m/z value. In such a case, two or more kinds of precursor ions are subjected to dissociation so that a product ion spectrum including peaks of product ions generated from the two or more kinds of precursor ions is obtained by measurement.

In this Variation, the first data generation part 522 acquires m/z values of two or more kinds of precursor ions. A mass resolution for the m/z values of the precursor ions is preferably 10 ppm or less, more preferably 1 ppm or less. These m/z values may be those of peaks included in a mass spectrum obtained by performing mass separation of precursor ions of the sample with the same settings as the first mass spectrometry and then scanning the mass-separated precursor ions without performing dissociation. Alternatively, the m/z values of peaks near the m/z value of precursor ions may be extracted from a mass spectrum obtained by single mass spectrometry of the sample. The m/z values of the two or more kinds of precursor ions obtained in such a manner are acquired by the mass spectrometry device 1 via the input section 41 or the communication section 42, or the mass spectrometry device 1 may perform such mass spectrometry.

The first data generation part 522 calculates two or more candidate structures C for each of the two or more kinds of precursor ions in the same manner as in the above-described embodiment. One of the two or more kinds of precursor ions is defined as a first precursor ion, and the other is defined as a second precursor ion. The candidate structures C of the first precursor ion are defined as first candidate structures, and the candidate structures C of the second precursor ion are defined as second candidate structures.

The second data generation part 523 generates candidate product data CP about the first candidate structures (referred to as first candidate product data) and candidate product data CP about the second candidate structures (referred to as second candidate product data) in the same manner as in the above-described embodiment. The second data generation part 523 combines the first candidate product data and the second candidate product data to generate combined candidate product data including at least one of the m/z values and the detected intensities of two or more kinds of product ions derived from the two or more kinds of precursor ions. The product ions overlapped with each other may appropriately be removed to generate, as the combined candidate product data, candidate product data including the m/z values or detected intensities of the product ions included in the two or more candidate product data to be combined.

The similarity calculation part 610 calculates a similarity between the combined candidate product data and the m/z values or detected intensities of detected product ions. The selection part 620 selects the combined candidate product data with the highest similarity, and the first candidate structure and the second candidate structure corresponding to the selected combined candidate product data are obtained as the structures of more than one kind of molecule contained in the sample.

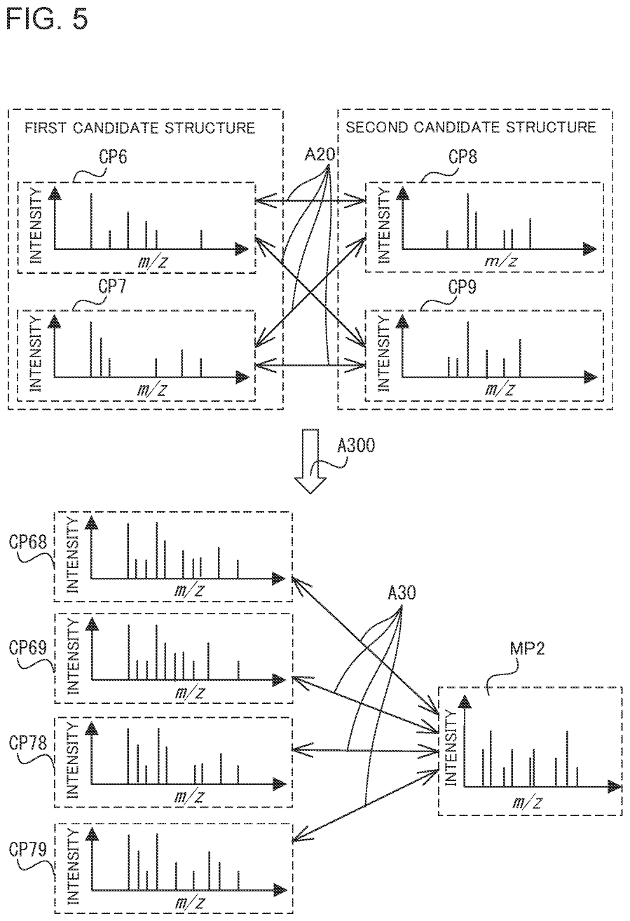

FIG. 5 is a conceptual diagram for explaining a method for analyzing data obtained by mass spectrometry according to the present Variation. The first data generation part 522 calculates first candidate structures and second candidate structures. The second data generation part 523 generates first candidate product data CP6 and CP7 corresponding to precursor ions having the first candidate structures. The second data generation part 523 generates second candidate product data CP8 and CP9 corresponding to precursor ions having the second candidate structures. The second data generation part 523 combines the first candidate product data CP6 or CP7 and the second candidate product data CP8 or CP9 to generate combined candidate product data (arrow A300). In FIG. 5, combinations of the two candidate product data to be combined are schematically shown by arrows A20. Combined candidate product data CP68, CP69, CP78, and CP79 correspond to a combination of the first candidate structure data CP6 and the second candidate structure data CP8, a combination of the first candidate structure data C6 and the second candidate structure data CP9, a combination of the first candidate structure data CP7 and the second candidate structure data CP8, and a combination of the first candidate structure data CP7 and the second candidate structure data CP8, respectively.

It is to be noted that the number of candidate structures, the number of candidate product data, and the number of combined candidate product data are not limited to those in the above example and may be set to any values.

The similarity calculation part 610 calculates a similarity between each of the combined candidate product data 68, 69, 78, and 79 and measured product data MP2 about detected product ions (arrow A30). The selection part 620 selects the combined candidate product data on the basis of the similarity, identifies the molecules contained in the sample, and creates an association between each kind of the product ions and each kind of the precursor ions.

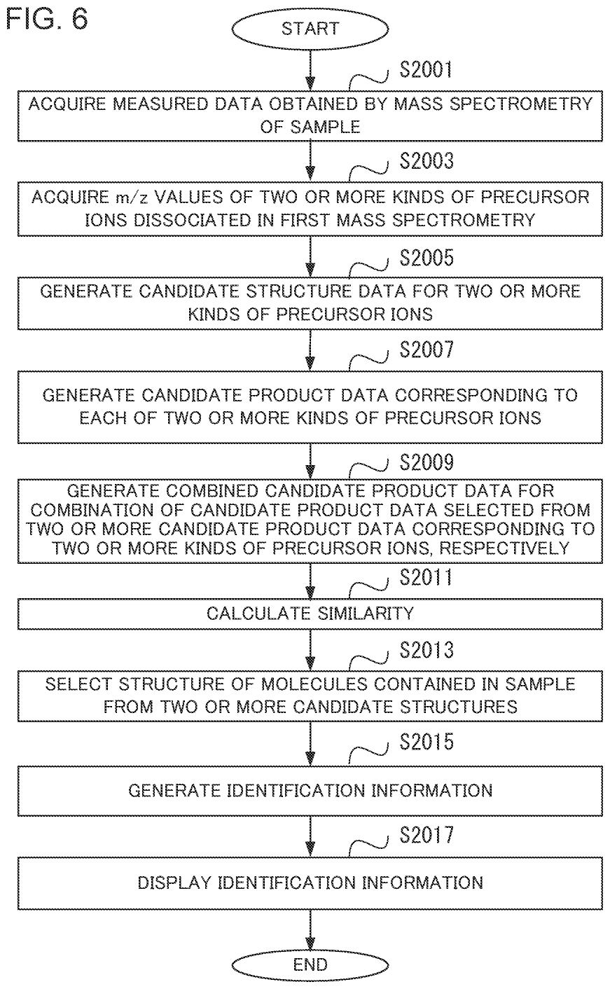

FIG. 6 is a flow chart showing the procedure of a method for analyzing data obtained by mass spectrometry according to the present Variation. Each step in FIG. 6 is appropriately performed by the control section 50. Step S2001 is the same as step S1001 in the above-described flow chart shown in FIG. 4, and therefore the description thereof is omitted. After step S2001 is complected, step S2003 is started.

In step S2003, the first data generation part 522 acquires the m/z values of two or more kinds of precursor ions dissociated in first mass spectrometry. After step S2003 is completed, step S2005 is started. In step S2005, the first data generation part 522 generates candidate structure data for the two or more kinds of precursor ions. After step S2005 is completed, Step S2007 is started.

In step S2007, the second data generation part 523 generates candidate product data corresponding to each of the two or more kinds of precursor ions. After step S2007 is completed, step S2009 is started. In step S2009, the second data generation part 523 generates combined candidate product data for a combination of the selected candidate product data corresponding to the two or more kinds of precursor ions respectively. After step S2009 is completed, step S2011 is started.

In step S2011, the similarity calculation part 610 calculates a similarity between the combined candidate product data and measured product data. After step S2011 is completed, step S2013 is started. Steps S2013 to S2017 are the same as steps S1009 to S1013 in the flow chart shown in FIG. 4, and therefore the description thereof is omitted.

The method according to the present Variation makes it possible, when the sample contains various molecules, such as when it is difficult to sufficiently perform pretreatment, to prevent a reduction in the accuracy of identification of molecules contained in the sample.

It is to be noted that, for example, when mass resolution is not sufficient in the first step of mass spectrometry for selecting precursor ions, there is a case where it is impossible to obtain the m/z value of each of the two or more kinds of precursor ions. In such a case, the first data generation part 522 can calculate, as candidate structures C, structures of a molecule having an m/z value within the selected range of m/z of a precursor ion or the range of m/z of a precursor ion appropriately estimated on the basis of measurement precision or the like. As described above with reference to Variation 3, the second data generation part 523 can generate combined candidate product data obtained by combining the calculated two or more candidate structures C. The processing after the calculation of similarity is the same as described above.

Variation 4

In the above-described Variation 3, the m/z values and detected intensity values of two or more kinds of precursor ions may be acquired to set, on the basis of the detected intensity values, the detected intensity values of product ions included in combined candidate product data.

The second data generation part 523 acquires relative values of detected intensities of two or more kinds of precursor ions. The relative value may be a detected intensity ratio between the two or more kinds of precursor ions or a detected intensity value of each of the two or more kinds of precursor ions standardized by the detected intensity of any peak in a spectrum including the peaks of the precursor ions. The second data generation part 523 can set the detected intensities of product ions corresponding to each of the candidate structures C by weighting based on the relative values when generating combined candidate product data.

For example, when the ratio between the detected intensity of a precursor ion corresponding to a first candidate structure and the detected intensity of a precursor ion corresponding to a second candidate structure is 1:2, the second data generation part 523 generates combined candidate product data by weighting the detected intensities of product ions in first candidate product data CP6 and CP7 and the detected intensities of product ions in second candidate product data CP8 and CP9 at a ratio of 1:2. This makes it possible to reflect information about the relative amounts of the precursor ions in information about the product ions as candidates, thereby more accurately identifying the molecule contained in the sample.

Variation 5

In the present Variation, when two or more kinds of precursor ions are mass-separated by mass separation of precursor ions in first mass spectrometry, an association between precursor ions and product ions is created on the basis of the compositions of the precursor ions and the compositions of the product ions.

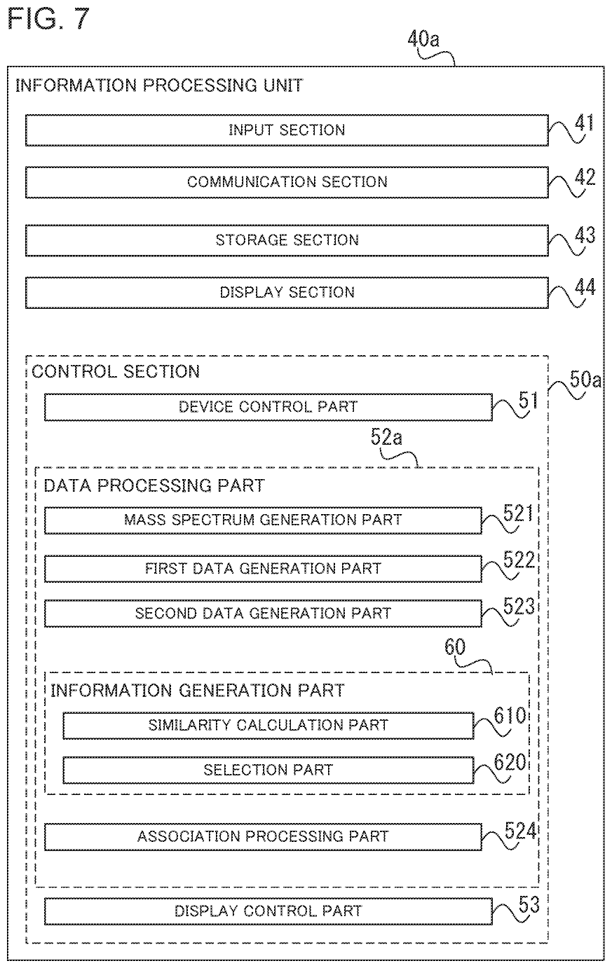

FIG. 7 is a conceptual diagram showing the structure of an information processing unit 40a according to the present Variation. The information processing unit 40a according to the present Variation includes a control section 50a having a data processing part 52a. The information processing unit 40a, the data processing part 52a, and the control section 50a are different from the information processing unit 40, the data processing part 52, and the control section 50 of the above-described embodiment in that an association processing part 524 is provided.

The association processing part 524 of the data processing part 52 generates information about an association between product ions and precursor ions for product ions detected in first mass spectrometry.

As described above with reference to Variation 3, the association processing part 524 acquires the m/z values of two or more kinds of the precursor ions. A mass resolution for the m/z value of the precursor ion is preferably 10 ppm or less, more preferably 1 ppm or less. The association processing part 524 calculates the composition of the precursor ions from the m/z value of the precursor ions. For example, the association processing part 524 can derive at least one of the kinds of elements contained in the precursor ion and the number of elements contained in the precursor ion. The association processing part 524 can calculate the composition of the precursor ion on the basis of sample information in addition to the m/z value of the precursor ion. In this case, the association processing part 524 may be configured to acquire, as sample information, information about elements contained in the sample via the input section 41 or the like.

The association processing part 524 calculates, from the m/z value of each kind of detected product ions, the composition of each kind of the product ions. For example, the association processing part 524 can derive at least one of the kinds of elements contained in the product ion and the number of elements contained in the product ion.

The association processing part 524 creates an association between precursor ions and product ions on the basis of the calculated compositions of the two or more kinds of precursor ions and the calculated compositions of the product ions. The association is created on the basis of, for example, the fact that an element contained in a product ion is contained also in a precursor ion. The creation of the association does not always need to be performed on the all kinds of product ions. For example, when a certain kind of product ions contain an element not contained in a certain kind of precursor ions, this kind of product ions are not product ions generated from this kind of precursor ions. In this case, the association processing part 524 can generate information showing such association and store it in the storage section 43 or the like. Alternatively, the association processing part 524 may generate measured product data for each of the two or more kinds of precursor ions so that the measured product data includes the m/z values or detected intensities of product ions that may correspond to the precursor ions. The information generation part 60 can calculate a similarity between each of the measured product data generated in this way and the candidate product data obtained in the above-described embodiment to perform identification on the basis of the similarity.

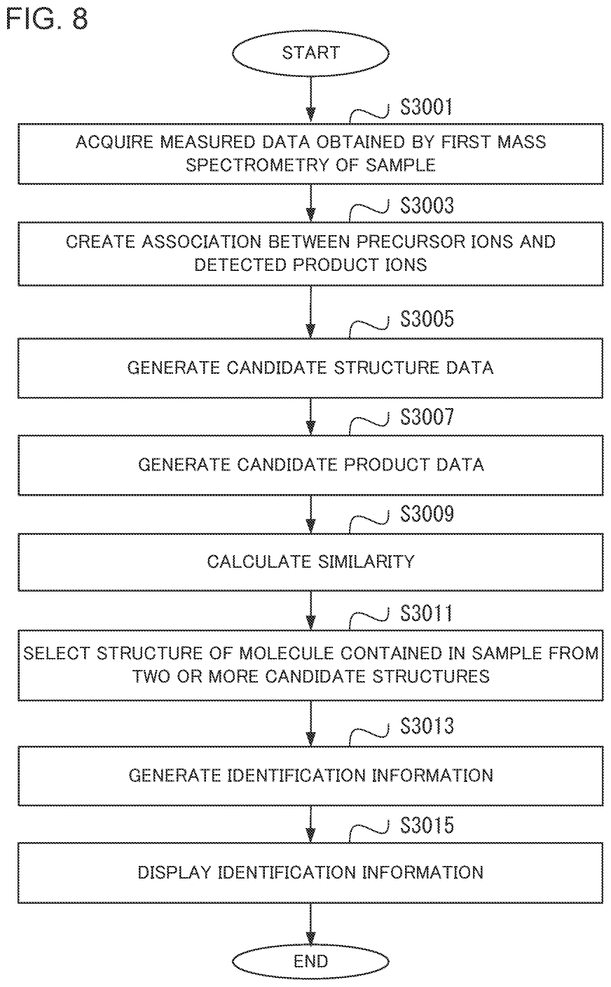

FIG. 8 is a conceptual diagram showing the procedure of a method for analyzing data obtained by mass spectrometry according to the present Variation. Each step in FIG. 8 is appropriately performed by the control section 50a. Step S3001 is the same as step S1001 in the flow chart shown in FIG. 4, and therefore the description thereof is omitted. After step S3001 is completed, step S3003 is started. In step S3003, the association processing part 524 creates an association between precursor ions and detected product ions. After step S3003 is completed, step S3005 is started. Steps S3005 to S3015 are the same as steps S1003 to 1013 in the above-described flow chart shown in FIG. 4, and therefore the description thereof is omitted.

It is to be noted that step S3003 may be performed after step S3005 or step S3007.

Variation 6

In the above-described embodiment, the mass spectrometry device 1 may be an imaging mass spectrometry device. In imaging mass spectrometry, various ions may be generated because sufficient pretreatment of a sample is not easy, and therefore, Variations 3 to 5 are particularly preferably applied.

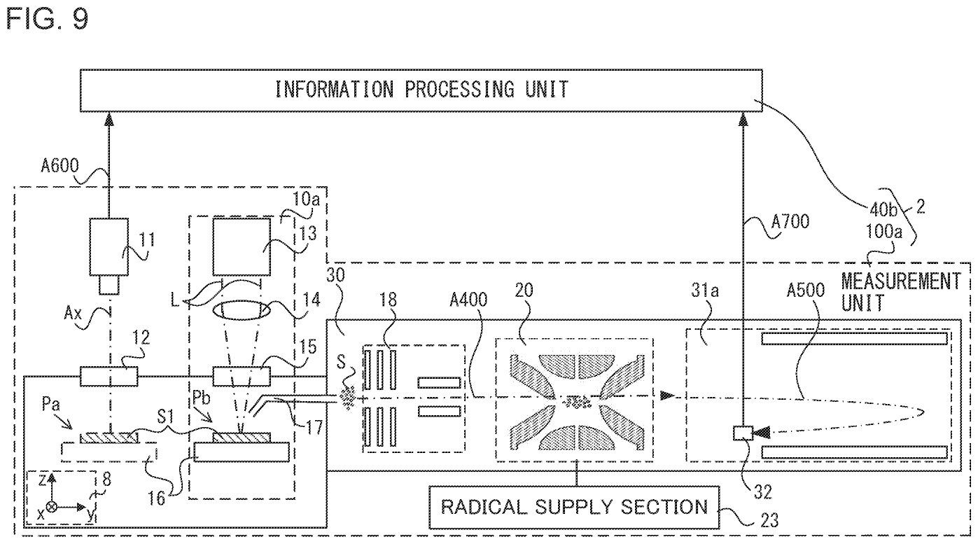

FIG. 9 is a conceptual diagram showing the structure of a mass spectrometry device 2 according to the present Variation. The mass spectrometry device 2 is an imaging mass spectrometry device, and includes a measurement unit 100a and an information processing unit 40b.

The measurement unit 100a includes an observation window 12, an imaging section 11 that takes an image of a sample 51 placed in a first position Pa through the observation window 12, an ionization section 10a that ionizes the sample 51 present in a second position Pb, an ion generation section 20, a radical supply section 23, a time-of-flight mass analysis section 31a, and a detection section 32. The ionization section 10a includes a laser 13, a condensing optical system 14, an irradiation window 15, a sample table 16, and an ion transport tube 17. In FIG. 9, a z-axis is set along an optical axis Ax of the imaging section 11, and an x-axis and a y-axis are set so as to be perpendicular to the z-axis (see a coordinate system 8).

The imaging section 11 includes an imaging device such as a camera. The sample table 16 is configured to be driven by a drive unit (not shown in the figure) so as to be movable between the first position Pa where the imaging section 11 can take an image of the sample S and the second position Pb where the sample S can be irradiated with laser light L from the laser 13. An irradiation area irradiated with the laser light L from the laser 13 is adjusted by the condensing optical system 14 including a lens. The position on the sample S1 where the laser light L is applied for ionization is referred to as an irradiation position. The ionization section 20 sequentially applies the laser light L onto each irradiation position to ionize a sample component present in the irradiation area corresponding to each irradiation position. Ions S derived from the sample generated by ionization by the ionization section 10a pass through the ion transport tube 17 and enter into the ion generation section 20 after the width of flux of the ions S is adjusted by an ion transport optical system 18 including a quadrupole or the like.

The ion generation section 20, the radical supply section 23, the time-of-flight mass analysis section 31a, and the detection section 32 can be configured in the same manner as those of the above-described embodiment, and therefore the description thereof is omitted. The voltage application section, the inert gas supply section, etc. shown in FIG. 1 are not shown in FIG. 9. The time-of-flight mass analysis section 31a shown in FIG. 9 is of a reflectron type, but its type is not particularly limited. In FIG. 9, the migration path of the ions S derived from the sample is shown by an arrow A400 and an arrow A500. The input of image data from the imaging section 11 to the information processing unit 40b is shown by an arrow A600. The input of measured data from the detection section 32 to the information processing unit 40b is shown by an arow A700.

The information processing unit 40b creates an association between the image of the sample S1 obtained by imaging of the imaging section 11 and the irradiation position. The information processing unit 40b is configured to be able to identify a molecule contained in the sample S1 from measured data corresponding to each irradiation position as in the case of the above-described embodiment.

Variation 7

A program for realizing an information processing function of the mass spectrometry device 1 and 2 can be recorded on a computer-readable recording medium, and a program related to control of processing of the data processing part 52, 52a and processing related thereto recorded on the recording medium may be loaded into a computer system and may be executed. It is noted that the term “computer system” in this context may refer to an OS (operating system) or a peripheral device in hardware. In addition, the “computer-readable recording medium” may be a portable recording medium such as a flexible disk, a magneto-optical disk, an optical disk or a memory card, or it may be a storage device such as a hard disk or a solid state drive built into the computer system. Furthermore, the “computer-readable recording medium” may be a medium that dynamically holds the program over a short period of time, e.g., a communication line through which the program is transmitted via a network such as the Internet or via a communication network such as a telephone network, or a medium that holds the program over a certain length of time, e.g., a volatile memory within a computer system functioning as a server or a client in the above case. Moreover, the program may allow only some of the functions described above to be fulfilled or the functions described above may be fulfilled by using the program in conjunction with a program pre-installed in the computer system.

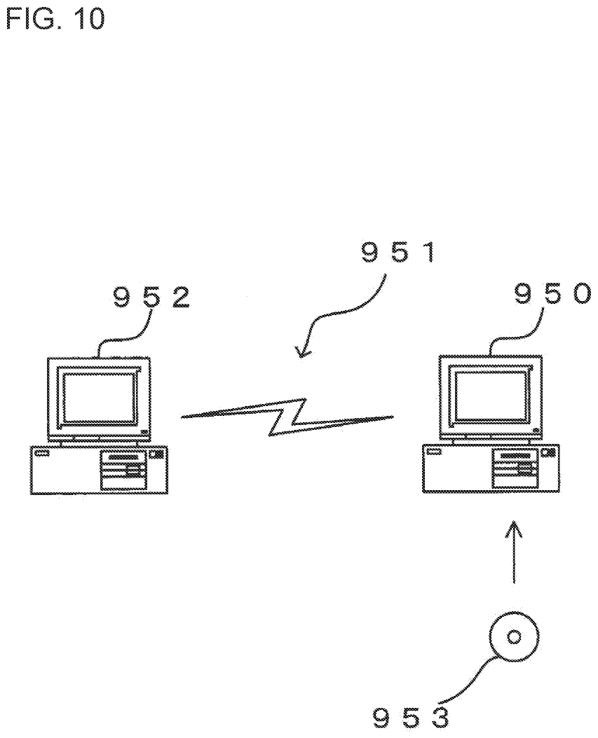

In addition, the present invention may be adopted in conjunction with a personal computer (hereafter referred to as a PC) or the like, and in such a case, the program pertaining to the control described above can be provided in a recording medium such as a DVD-ROM or on a data signal transmitted through the Internet or the like. FIG. 10 illustrates how such a program may be provided. A PC 950 receives the program via a CD-ROM 953. The PC 950 is also capable of connecting with a communication network 951. A computer 952 is a server computer that provides the program stored in a recording medium such as a hard disk. The communication network 951 may be a communication network such as the Internet or a personal computer communication network, or it may be a dedicated communication network. The computer 952 reads out the program from the hard disk and transmits it to the PC 950 via the communication network 951. In other words, the program may be delivered as a data signal carried on a carrier wave transmitted via the communication network 951. Namely, the program can be distributed as a computer-readable computer program product assuming any of various modes including a recording medium and a carrier wave.

Aspects

It is understood by those skilled in the art that the above-described illustrative embodiments or variations thereof are specific examples of the following aspects.

Item 1

A device for analyzing data obtained by mass spectrometry according to one aspect includes a data acquisition section that acquires mass spectrometry data obtained by first mass spectrometry including subjecting a precursor ion derived from a sample to a reaction using a radical, a first data generation section that generates, on a basis of sample information about a molecule contained in the sample, first data showing two or more candidate structures of the molecule or the precursor ion, a second data generation section that generates, on a basis of the first data and data showing a condition of the reaction, second data showing at least one of a mass-to-charge ratio and a detected intensity of a product ion generated in a case of subjecting the precursor ion having each of the structures to the reaction, and an information generation section that generates, on a basis of at least one of a mass-to-charge ratio and a detected intensity of a detected product ion in the mass spectrometry data and the second data, information about identification of the molecule contained in the sample. This makes it possible to analyze the structure of the molecule contained in the sample in more detail on the basis of the characteristics of the reaction using a radical.

Item 2

A device for analyzing data obtained by mass spectrometry according to another aspect is the device for analyzing data obtained by mass spectrometry according to Item 1, wherein when the sample contains a lipid, the first data generation section generates the first data showing the candidate structures different in at least one of a length of a carbon chain in a fatty acid contained in the lipid, a position of a double bond, and a cis-trans type of the double bond. This makes it possible to analyze the structure concerning a carbon chain and a double bond of the molecule contained in the sample in more detail.

Item 3

A device for analyzing data obtained by mass spectrometry according to another aspect is the device for analyzing data obtained by mass spectrometry according to Item 1 or Item 2, wherein the sample information includes information obtained by two- or more-step second mass spectrometry including dissociation of the molecule contained in the sample by collision-induced dissociation or dissociation using a radical. This makes it possible to more accurately perform mass spectrometry from information obtained by the first mass spectrometry and the second mass spectrometry.

Item 4

A device for analyzing data obtained by mass spectrometry according to another aspect is the device for analyzing data obtained by mass spectrometry according to any one of Item 1 to Item 3, wherein the sample information includes a kind of the molecule contained in the sample, and when the molecule is a lipid, the sample information includes at least one of a class or a head group of a lipid, a length of a carbon chain of a fatty acid contained in a lipid, a number of double bonds in the carbon chain, and a modification group in the carbon chain. This makes it possible to more efficiently analyze the structure of the lipid contained in the sample.

Item 5

A device for analyzing data obtained by mass spectrometry according to another aspect is the device for analyzing data obtained by mass spectrometry according to any one of Item 1 to Item 5, wherein in the first mass spectrometry, the precursor ion is subjected to a reaction with at least one of a hydrogen radical, a hydroxy radical, a nitrogen radical, and an oxygen radical, the second data generation section generates, on a basis of the first data and the data showing a condition of the reaction, the second data showing at least one of a mass-to-charge ratio and a detected intensity of the product ion generated in a case of subjecting the precursor ion to the reaction with at least one of a hydrogen radical, a hydroxy radical, a nitrogen radical, and an oxygen radical. This makes it possible to analyze the structure of the molecule contained in the sample in more detail on the basis of the characteristics of a hydrogen radical, a hydroxy radical, a nitrogen radical, or an oxygen radical.

Item 6

A device for analyzing data obtained by mass spectrometry according to another aspect is the device for analyzing data obtained by mass spectrometry according to Item 5, wherein in the first mass spectrometry, a radical is generated using, as a raw material gas, at least one of nitrogen, oxygen, hydrogen, hydrogen peroxide, and water vapor. These raw material gases are available without difficulty, and the structure of the molecule contained in the sample can more efficiently be analyzed.

Item 7

A device for analyzing data obtained by mass spectrometry according to another aspect is the device for analyzing data obtained by mass spectrometry according to any one of Item 1 to Item 6, wherein the second data generation section generates, on a basis of the first data and the data showing a condition of the reaction, the second data showing at least one of charge-to-mass ratios and detected intensities of fragment ions generated in the case of subjecting the precursor ions to the reaction, and the information generation section includes an information generation part that generates, on a basis of at least one of mass-to-charge ratios and detected intensities of detected ions in the mass spectrometry data and the second data, information about at least one of a length of a carbon chain in the molecule contained in the sample and a position of a double bond in the carbon chain. This makes it possible to analyze the structure of the molecule contained in the sample in more detail on the basis of the characteristics of dissociation using a radical.

Item 8

A device for analyzing data obtained in mass spectrometry according to another aspect is the device for analyzing data obtained in mass spectrometry according to any one of Item 1 to Item 7, wherein the second data generation section generates, on a basis of the first data and the data showing a condition of the reaction, the second data showing relative detected intensities of two or more kinds of adduct ions generated by subjecting the precursor ions to the reaction, and the information generation section generates, on a basis of a detected intensity of detected adduct ions in the mass spectrometry data and the second data, information about cis-trans isomers of the molecule contained in the sample. This makes it possible to analyze the structure of the molecule contained in the sample in more detail on the basis of the characteristics of addition, attachment, or the like using a radical.

Item 9

A device for analyzing data obtained by mass spectrometry according to another aspect is the device for analyzing data obtained by mass spectrometry according to any one of Item 1 to Item 8, wherein the first data generation section generates the first data for each of the two or more kinds of molecules different in mass or each of the two or more kinds of precursor ions different in mass-to-charge ratio, the second data generation section generates, for each combination of the two or more kinds of precursor ions different in mass-to-charge ratio, the second data showing at least one of mass-to-charge ratios and detected intensities of product ions generated by subjecting the precursor ions included in the combination to the reaction, and the information generation section generates information about identification of the two or more kinds of molecules contained in the sample. This makes it possible to analyze the structures of the molecules contained in the sample in more detail on the basis of the characteristics of the reaction using a radical even when the two or more kinds of precursor ions are subjected to the radical reaction.

Item 10

A device for analyzing data obtained by mass spectrometry according to another aspect is the device for analyzing data obtained by mass spectrometry according to Item 9, wherein the second data generation section sets detected intensities of the product ions included in the second data on a basis of relative values of detected intensities of the two or more kinds of precursor ions different in mass-to-charge ratio. This makes it possible to analyze the structures of the molecules contained in the sample in more detail when the two or more kinds of precursor ions are subjected to the radical reaction.

Item 11

A device for analyzing data obtained by mass spectrometry according to another aspect is the device for analyzing data obtained by mass spectrometry according to any one of Item 1 to Item 8, including an association processing section that derives, on a basis of a mass-to-charge ratio of the precursor ion and a mass-to-charge ratio of the product ion, a composition of the precursor ion and a composition of the product ion and generates, on a basis of the compositions, information as to whether the detected two or more kinds of the product ions correspond to the precursor ions. This makes it possible, even when the sample contains various molecules, such as when the sample contains foreign matter, to prevent a reduction in the accuracy of analysis of the structure of the molecule contained in the sample.

Item 12

A mass spectrometry device according to one aspect includes a measurement unit that performs first mass spectrometry including subjecting a precursor ion derived from a sample to a reaction using a radical, and the device for analyzing data obtained by mass spectrometry according to any one of Item 1 to Item 11. This makes it possible to analyze the structure of molecule contained in the sample in more detail on the basis of the characteristics of the reaction using a radical.

Item 13

A method for analyzing data obtained by mass spectrometry according to one aspect includes acquiring mass spectrometry data obtained by first mass spectrometry including subjecting a precursor ion derived from a sample to a reaction using a radical, generating, on a basis of sample information about a molecule contained in the sample, first data showing two or more candidate structures of the molecule or the precursor ion, generating, on a basis of the first data and data showing a condition of the reaction, second data showing at least one of a mass-to-charge ratio and a detected intensity of a product ion generated in a case of subjecting the precursor ion having each of the structures to the reaction, and generating, on a basis of at last one of a mass-to-charge ratio and a detected intensity of a detected product ion in the mass spectrometry data and the second data, information about identification of the molecule contained in the sample. This makes it possible to analyze the structure of the molecule contained in the sample in more detail on the basis of the characteristics of the reaction using a radical.

Item 14

An analysis program according to one aspect enables a processing device to perform data acquisition processing (corresponding to step S1001 in the flow chart shown in FIG. 4) to acquire mass spectrometry data obtained in first mass spectrometry including subjecting a precursor ion derived from a sample to a reaction using a radical, first data generation processing (corresponding to step S1003) to generate, on a basis of sample information about a molecule contained in the sample, first data showing two or more candidate structures of the molecule or the precursor ion, second data generation processing (corresponding to step S1005) to generate, on a basis of the first data and data showing a condition of the reaction, second data showing at least one of a mass-to-charge ratio and a detected intensity of a product ion generated in case of subjecting the precursor ion having each of the structures to the reaction, and information generation processing (corresponding to step S1011) to generate, on a basis of at least one of a mass-to-charge ratio and a detected intensity of a detected product ion in the mass spectrometry data and the second data, information about identification of the molecule contained in the sample. This makes it possible to analyze the structure of the molecule contained in the sample in more detail on the basis of the characteristics of the reaction using a radical.

The present invention is not limited to the contents of the above-described embodiments, and other aspects conceivable within the scope of the technical idea of the present invention are also included in the scope of the present invention.

### EXAMPLES

Hereinbelow, an Example according to the present embodiment will be described, but the present invention is not intended to be limited by the following Example.

Two-step mass spectrometry including dissociation by CID was performed on a known sample containing phosphatidylcholine (PC (16/20:4(5Z, 8Z, 11Z, 14Z))). The sample was assumed as an unknown sample, and from the results of the mass spectrometry, the precise mass of a precursor ion corresponding to the phosphatidylcholine, the length of a carbon chain of a fatty acid, and the number of double bonds were derived. From the accurate mass, the length of a carbon chain, and the number of double bonds as conditions, all the candidate structures conceivable as molecules contained in the sample were calculated. From the calculated candidate structures, a product ion spectra when the precursor ions corresponding to each of the candidate structures were dissociated using hydroxy radicals and using hydrogen radicals were calculated.

Two-step mass spectrometry including dissociation using hydroxy radicals was performed on the above-described sample. A sample prepared by diluting 100 pmol of the above-described sample with methanol was used. Ionization was performed by MALDI, selection and dissociation of precursor ions corresponding to the phosphatidylcholine were performed in an ion trap, and then obtained fragment ions were mass-separated by time-of-flight mass spectrometry and detected. The hydroxy radicals were generated by water vapor discharge, and the precursor ions were irradiated with the hydroxy radicals for a reaction period of 100 ms.

Further, two-step mass spectrometry including dissociation using hydrogen radicals was performed on the above-described sample. The conditions of the mass spectrometry were the same as those in the case where hydroxy radicals were used. The hydrogen radicals were generated by thermal dissociation of hydrogen gas, and the precursor ions were irradiated with the hydrogen radicals for a reaction period of 500 ms.

A mass spectrum was calculated by merging the product ion spectrum of the fragment ions obtained by hydroxy radicals and the product ion spectrum of the fragment ions obtained by hydrogen radicals. A similarity between this mass spectrum and the above-described product ion spectrum calculated from each of the candidate structures was calculated using software (NIST MS Search).

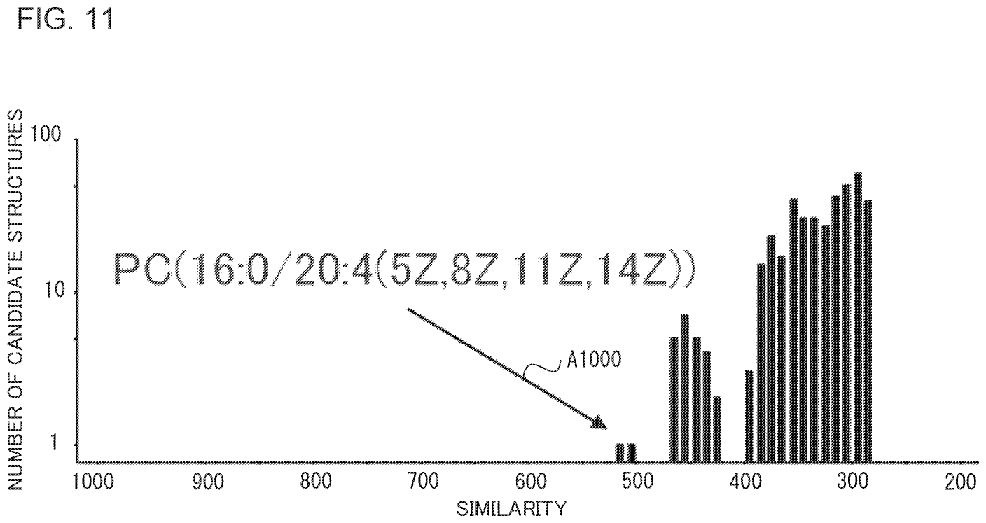

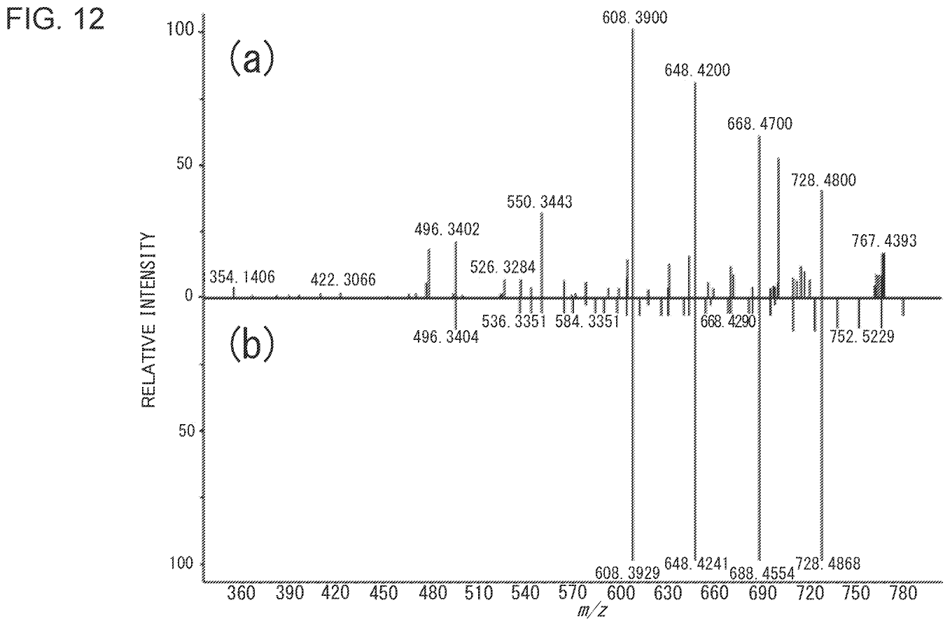

FIG. 11 is a graph in which a horizontal axis represents a calculated similarity and a vertical axis represents the number of candidate structures corresponding to the similarity. There was one candidate structure having the highest similarity (arrow A1000), and this candidate structure corresponded to the phosphatidylcholine actually contained in the sample (PC(16/20.4(5Z, 8Z, 11Z, 14Z))). (a) in FIG. 12 shows a mass spectrum obtained by measurement in the present example, and (b) in FIG. 12 shows one of the product ion spectra corresponding to the calculated candidate structures and having the highest similarity. In the graph (a) in FIG. 12, a horizontal axis represents the m/z of detected ions and a vertical axis represents the relative intensity of detection signal of each ion with respect to the peak intensity of the precursor ion. In the graph (b) in FIG. 12, a horizontal axis represents the m/z of calculated fragment ions, and a vertical axis represents the relative intensity of detection signal of calculated fragment ions.

### REFERENCE SIGNS LIST

1, 2 Mass spectrometry device
10, 10a Ionization section
20 Ion generation section
22 Inert gas supply section
23 Radical supply section
31, 31a Time-of-flight mass separation section
32 Detection section
40 Information processing unit
43 Storage section
44 Display section
50 Control section
51 Device control part
52 Data processing part
53 Display control part
60 Information generation part
100 Measurement unit
211, 212 End cap electrode
213 Ring electrode
216 Radical introduction part
230 Raw material gas supply source
232 Radical generation part
233 Radio-frequency supply part
521 Mass spectrum generation part
522 First data generation part
523 Second data generation part
524 Association processing part
610 Similarity calculation part
620 Selection part
C, C1, C2, C3, C4, C5 Candidate structure
CP, CP1, CP2, CP3, CP4, CP5 Candidate product data
CP6, CP7 First candidate product data
CP8, CP9 Second candidate product data
CP8, CP9, CP68, CP69, CP78, CP79 Combined candidate product data
MP1, MP2 Measured product data
S Sample-derived ion
S1 Sample

1. A device for analyzing data obtained by mass spectrometry, comprising:
a data acquisition section that acquires mass spectrometry data obtained by first mass spectrometry including subjecting a precursor ion derived from a sample to a reaction using a radical;
a first data generation section that generates, on a basis of sample information about a molecule contained in the sample, first data showing two or more candidate structures of the molecule or the precursor ion;
a second data generation section that generates, on a basis of the first data and data showing a condition of the reaction, second data showing at least one of a mass-to-charge ratio and a detected intensity of a product ion generated in a case of subjecting the precursor ion having each of the structures to the reaction; and
an information generation section that generates, on a basis of at least one of a mass-to-charge ratio and a detected intensity of a detected product ion in the mass spectrometry data and the second data, information about identification of the molecule contained in the sample.

2. The device for analyzing data obtained by mass spectrometry according to claim 1, wherein:
when the sample contains a lipid, the first data generation section generates the first data showing the two or more candidate structures different in at least one of a length of a carbon chain of a fatty acid contained in the lipid, a position of a double bond, and a cis-trans type of the double bond.

3. The device for analyzing data obtained by mass spectrometry according to claim 1, wherein:
the sample information includes information obtained by two- or more-step second mass spectrometry including dissociation of the molecule contained in the sample by collision-induced dissociation or dissociation using a radical.

4. The device for analyzing data obtained by mass spectrometry according to claim 1, wherein:
the sample information includes a kind of the molecule contained in the sample; and
when the molecule is a lipid, the sample information includes at least one of a class or a head group of a lipid, a length of a carbon chain of a fatty acid contained in a lipid, a number of double bonds in the carbon chain, and a modification group in the carbon chain.

5. The device for analyzing data obtained by mass spectrometry according to claim 1, wherein:
in the first mass spectrometry, the precursor ion is subjected to a reaction with at least one of a hydrogen radical, a hydroxy radical, a nitrogen radical, and an oxygen radical; and
the second data generation section generates, on a basis of the first data and the data showing a condition of the reaction, the second data showing at least one of a mass-to-charge ratio and a detected intensity of the product ion generated in a case of subjecting the precursor ion to the reaction with at least one of a hydrogen radical, a hydroxy radical, a nitrogen radical, and an oxygen radical.

6. The device for analyzing data obtained by mass spectrometry according to claim 5, wherein:
in the first mass spectrometry, a radical is generated using, as a raw material gas, at least one of nitrogen, oxygen, hydrogen, hydrogen peroxide, and water vapor.

7. The device for analyzing data obtained by mass spectrometry according to claim 1, wherein:
the second data generation section generates, on a basis of the first data and the data showing a condition of the reaction, the second data showing at least one of mass-to-charge ratios and detected intensities of fragment ions generated in a case of subjecting the precursor ion to the reaction; and
the information generation section generates, on a basis of at least one of mass-to-charge ratios and detected intensities of detected ions in the mass spectrometry data and the second data, information about at least one of a length of a carbon chain in the molecule contained in the sample and a position of a double bond in the carbon chain.

8. The device for analyzing data obtained by mass spectrometry according to claim 1, wherein:
the second data generation section generates, on a basis of the first data and the data showing a condition of the reaction, the second data showing relative detected intensities of two or more kinds of adduct ions generated by subjecting the precursor ion to the reaction; and
the information generation section generates, on a basis of detected intensities of detected adduct ions in the mass spectrometry data and the second data, information about cis-trans isomers of the molecule contained in the sample.

9. The device for analyzing data obtained by mass spectrometry according to claim 1, wherein:
the first data generation section generates the first data for each of the two or more kinds of molecules different in mass or each of the two or more kinds of precursor ions different in mass-to-charge ratio; and
the second data generation section generates, for each combination of the two or more kinds of precursor ions different in mass-to-charge ratio, the second data showing at least one of mass-to-charge ratios and detected intensities of product ions generated by subjecting the precursor ions included in the combination to the reaction; and
the information generation section generates information about identification of the two or more kinds of molecules contained in the sample.

10. The device for analyzing data obtained by mass spectrometry according to claim 9, wherein:
the second data generation section sets detected intensities of the product ions included in the second data on a basis of relative values of detected intensities of the two or more kinds of precursor ions different in mass-to-charge ratio.

11. The device for analyzing data obtained by mass spectrometry according to claim 1, comprising:
an association processing section that derives, on a basis of a mass-to-charge ratio of the precursor ion and a mass-to-charge ratios of the product ion, a composition of the precursor ion and a composition of the product ion, and generates, on a basis of the compositions, information as to whether the detected two or more kinds of the product ions correspond to the precursor ion.

12. A mass spectrometry device comprising:
a measurement unit that performs first mass spectrometry including subjecting a precursor ion derived from a sample to a reaction using a radical; and
the device for analyzing data obtained by mass spectrometry according to claim 1.

13. A method for analyzing data obtained by mass spectrometry, comprising:
acquiring mass spectrometry data obtained by first mass spectrometry including subjecting a precursor ion derived from a sample to a reaction using a radical;
generating, on a basis of sample information about a molecule contained in the sample, first data showing two or more candidate structures of the molecule or the precursor ion;
generating, on a basis of the first data and data showing a condition of the reaction, second data showing at least one of a mass-to-charge ratio and a detected intensity of a product ion generated in a case of subjecting the precursor ion having each of the structures to the reaction; and
generating, on a basis of at least one of a mass-to-charge ratio and a detected intensity of a detected product ion in the mass spectrometry data and the second data, information about identification of the molecule contained in the sample.

14. A computer-readable computer program product containing an analysis program for allowing a computer to perform:
data acquisition processing to acquire mass spectrometry data obtained by first mass spectrometry including subjecting a precursor ion derived from a sample to a reaction using a radical;
first data generation processing to generate, on a basis of sample information about a molecule contained in the sample, first data showing two or more candidate structures of the molecule or the precursor ion;
second data generation processing to generate, on a basis of the first data and data showing a condition of the reaction, second data showing at least one of a mass-to-charge ratio and a detected intensity of a product ion generated in a case of subjecting the precursor ion having each of the structures to the reaction; and
information generation processing to generate, on a basis of at least one of a mass-to-charge ratio and a detected intensity of a detected product ion in the mass spectrometry data and the second data, information about identification of the molecule contained in the sample.
